(19) United States
(12) Patent Application Publication
(10) Pub. No.: US 2006/0184223 A1
(43) Pub. Date: Aug. 17, 2006

(54) Cavity probe with exciter and/or dilator tip

(76) Inventor: John B. Squicciarini, Aliso Viejo, CA (US)

Correspondence Address: FISH & RICHARDSON, PC, P.O. BOX 1022, MINNEAPOLIS, MN, 55440-1022, US

(21) Appl. No.: 11/348,976

(22) Filed: Feb. 6, 2006

Related U.S. Application Data
(60) Provisional application No. 60/650,060, filed on Feb. 4, 2005.

(51) Int. Cl. A61N 1/05

(52) U.S. Cl. 607/138

(57) ABSTRACT

A probe for a mammalian orifice or cavity is presented. The probe includes a video probe to record video and/or images of the internal cavity area. Further, the device includes a mechanism by which fluids, tissue, or other samples can be accurately collected for later laboratory analysis. The probe further includes an electronic tissue exciter to promote physiological responses to electromagnetic stimuli. The probe also includes a dilator to expand and open an orifice or cavity to improve a range and depth of view of the tissue therein.

### CROSS REFERENCE TO RELATED APPLICATIONS

The present application claims priority under 35 U.S.C. §119 to U.S. Provisional Application Ser. No. 60/650,060, filed Feb. 4, 2005, entitled CAVITY PROBE WITH EXCITER AND/OR DILATOR TIP, the disclosure of which is incorporated herein by reference.

### BACKGROUND

Examination of a mammalian orifice or cavity, such as a vagina or cervix, is presently a very arduous and/or invasive procedure. Likewise, conventional examination techniques and devices are neither accurate enough to spot anomalies in a stage early enough to be beneficial, nor configured to promote natural physiological responses by tissue in order to emphasize or highlight such anomalies. Finally, in order to get an effective examination of an orifice or cavity, conventional techniques and devices involve very long and overly costly examinations, which can deter a medical practitioner and/or patient from doing them in the first place.

### SUMMARY

This document discloses an improved cavity probe. The probe includes a video probe to record video and/or images of the internal cavity area. Further, the device includes a mechanism by which fluids, tissue, or other samples can be accurately collected for later laboratory analysis. In one embodiment, the probe includes an electronic tissue exciter to promote physiological responses to electromagnetic stimuli. In another embodiment, the probe includes a dilator to expand and open an orifice or cavity to improve a range and depth of view of the tissue therein.

The details of one or more embodiments are set forth in the accompanying drawings and the description below. Other features and advantages will be apparent from the description and drawings, and from the claims.

### BRIEF DESCRIPTION OF THE DRAWINGS

These and other aspects will now be described in detail with reference to the following drawings.

FIG. 1 shows a probe with an exciter tip.

FIGS. 2A-C illustrate the exciter tip.

FIG. 3 shows a probe with an exciter tip and collection device.

FIGS. 4A and B and FIG. 5 illustrate a probe with a dilator mechanism in accordance with one embodiment.

FIGS. 6-10 illustrate a probe with a dilator mechanism in accordance with another embodiment.

Like reference symbols in the various drawings indicate like elements.

### DETAILED DESCRIPTION

This document describes a device that easily and comfortably spreads and holds back tissue of a cavity of a mammal, such as a cervix or vagina.

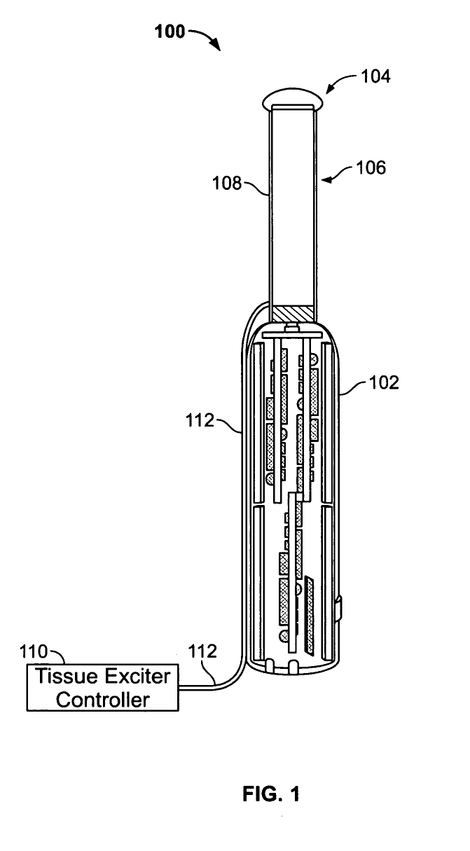

FIG. 1 shows a probe 100 having a handle 102 connected to a tip 104. The probe 100 can physically compact. The tip 104 includes a shaft 106 that is sized and configured for insertion into an orifice or cavity of a mammal. The tip 104 can include a video camera. An exciter sheath 108 is provided to the surface of the shaft 106 and/or tip 104 and is configured to electrically stimulate mammalian tissue inside the orifice or cavity. The exciter sheath 108 can be wrapped around the shaft 106 and/or tip 104, or affixed to the shaft 106 and/or tip 104. One example of the exciter sheath 108 is a transcutaneous electrical nerve stimulation (TENS) pad material having one or more electrodes to administer an electrical current at a particular frequency. Alternatively, the exciter sheath 108 can deliver vibration energy.

The exciter sheath 108 is controlled by a tissue exciter controller 110 that sends control signals via control path 112. The tissue exciter controller 110 may include a built-in microprocessor, or control signals may be generated externally and sent to the tissue exciter controller 110 for delivery to the exciter sheath 108. The control path 112 may be either a physical electrical connection, or a wireless communication path. In one embodiment, the control path 112 includes a wire connected to a lower portion of the exciter sheath 108 from along a side of the handle 102 of the probe 100. In an alternative embodiment, the tissue exciter controller 110 is built into the handle 102, and can include a number of user-selectable control buttons or similar devices.

The exciter sheath 108 electrically stimulates tissue such as muscle or other surface or subsurface tissue within the orifice or cavity, thereby generating physiological responses such as quivering or pulsating, and accentuating physiological conditions such as subsurface abnormalities. Thus, abnormalities can be brought to the surface inside of an orifice or cavity. Also, by using the probe 100 to closely monitor tissue texture, color, elasticity, etc., infection, disease, lesions, and/or other abnormalities may be detected sooner than with conventional methods, or before they are visible on the inside surface of the orifice or cavity. Accordingly, invasive sampling or examination can be avoided.

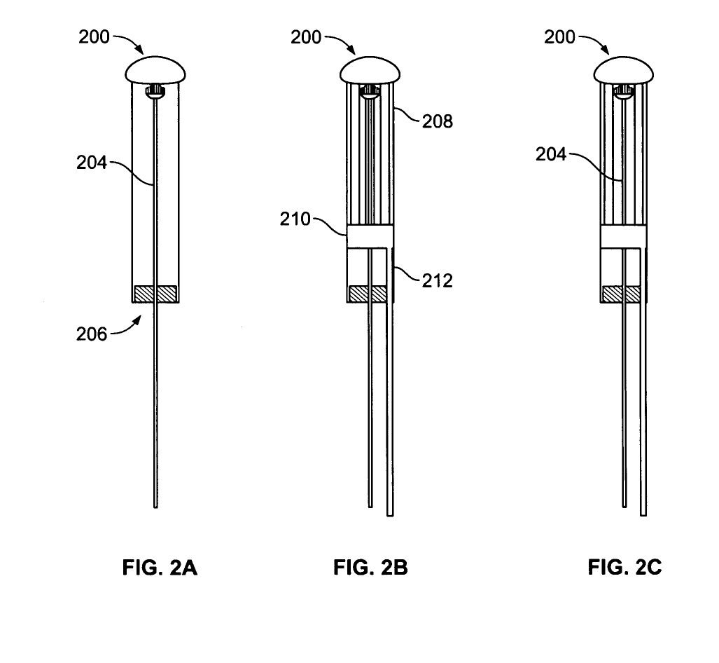

FIGS. 2A-C illustrate a probe tip 200 that is assembled for spreading and holding back tissue within a orifice or cavity of a mammal, such as a cervix. FIG. 2A shows a basic probe tip 200 that encloses at least one collection device 204 connected with a collector handle 206. The collection device 204 can be tucked within molded cavities inside the tip 200 to prevent contamination of obtained tissue samples. The tip 200 may also include a video and/or photographic collection device.

FIG. 2B shows the tip 200 at least partially encompassed by a dilator 208. The dilator 208 includes one or more slatted extensions configured for being movable from a closed position resting against the tip to an open position extending outwardly from the tip 200. The dilator 208 can be secured to the tip at a non-extending end by a sleeve 208, which can be slidably mounted onto or over the tip 200 and dilator non-extending end. The dilator 208 is controlled by and coupled to a dilator control handle 212. An expandable mesh material 214 can be provided around dilator 208, as shown in FIG. 2C, to prevent pinching surfaces to protect tissue being examined, and to further inhibit the collection devices 204 from contacting tissue when not required.

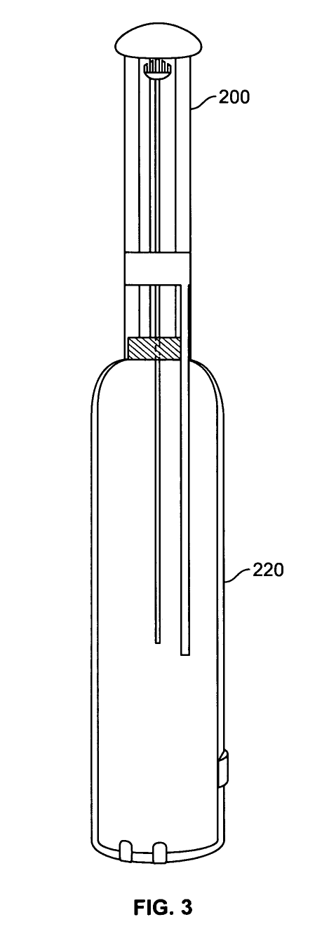

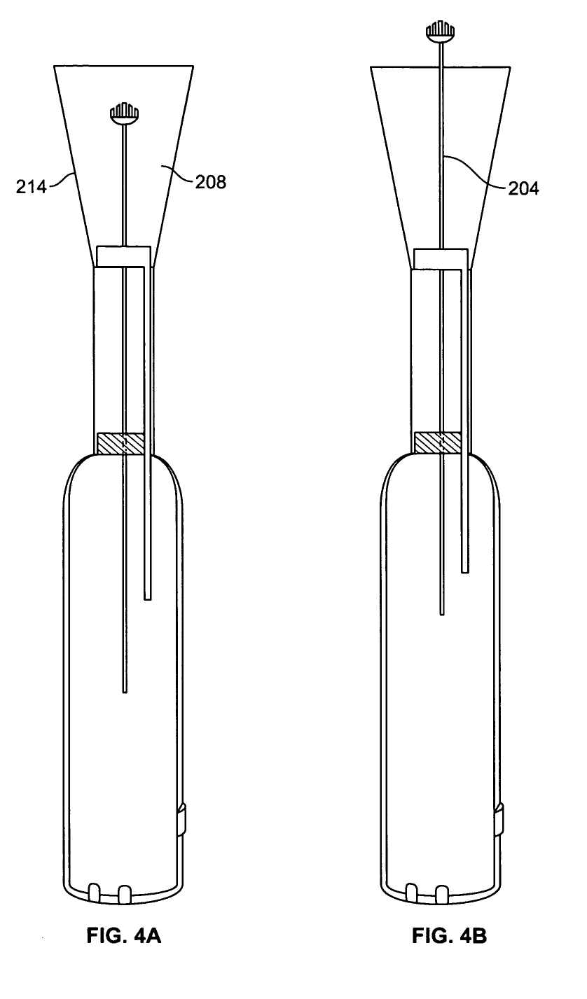

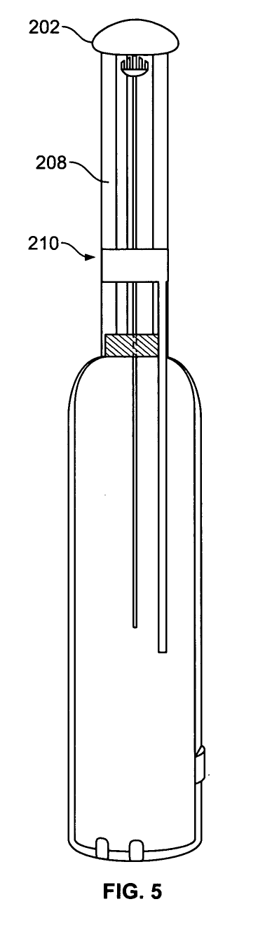

FIG. 3 illustrates the tip 200 coupled to a handle 220. As shown in FIG. 4A, in operation the tip 200 is inserted into an orifice or cavity with the dilator 208 in the closed position. Once inserted, when the tip 200 is retracted, the slatted extensions of the dilator 208 begin to spread outward toward the open position, encased by the elastic mesh 214, and spreading and holding back tissue for an expanded opening from the top of the tip 200. The opened dilator 208 can be held in place by the dilator control handle 212. As shown in FIG. 4B, the sampling device 204 can extended to collect sample tissue or fluids from the expanded orifice or cavity, and then retracted into the tip 200 to protect against contamination or damage.

The expansion of the dilator 208 can be calibrated or limited such that when adequate pressure is reached to comfortably hold back tissue, the dilator 208 material will start bending to maintain that pressure, while not overextending the tissue's expansion limits. Thus, discomfort by the mammal can be minimized. As shown in FIG. 5, the dilator 208 can be closed when the tip 200 is pushed back into the dilator 208, and can be locked back into the closed position once the tip 200 is completely within the dilator 208. Then, the entire probe can be safely and comfortably removed from the orifice or cavity, and the collected sample can be removed from the shielded collection device 204 for transport and analysis.

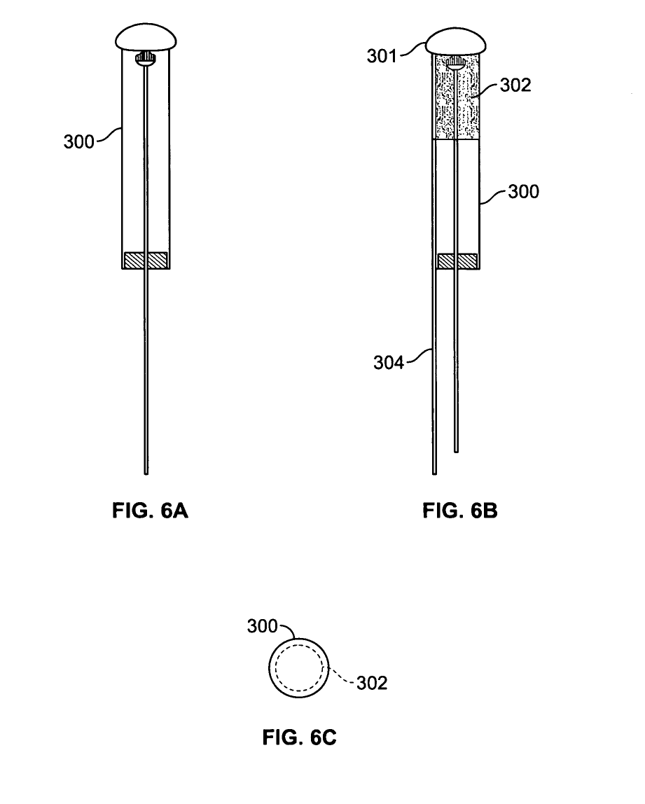

FIGS. 6A and B show a probe tip 300 and an alternative embodiment of a dilator. In FIG. 6A, the probe tip 300 includes a collection device 303 for collecting samples of tissue or other items within an orifice or cavity of a mammal. The collection device 303 can tucked into a molded cavity within a shaft of the tip 300, and extended and retracted, or otherwise controlled, by a handle. In FIG. 6B, the probe tip 300 includes a dilator 302 provided to and at least partially circumscribing the tip 300. In an embodiment, the dilator 302 is made of an expandable latex or rubber-based material, in a form of a balloon, and is enfolded around the top 301 of the tip 300. The dilator 302 is connected to a conduit 304. FIG. 6C shows a top-down cross sectional view of the top 301, which can be a lens or other soft-shaped end.

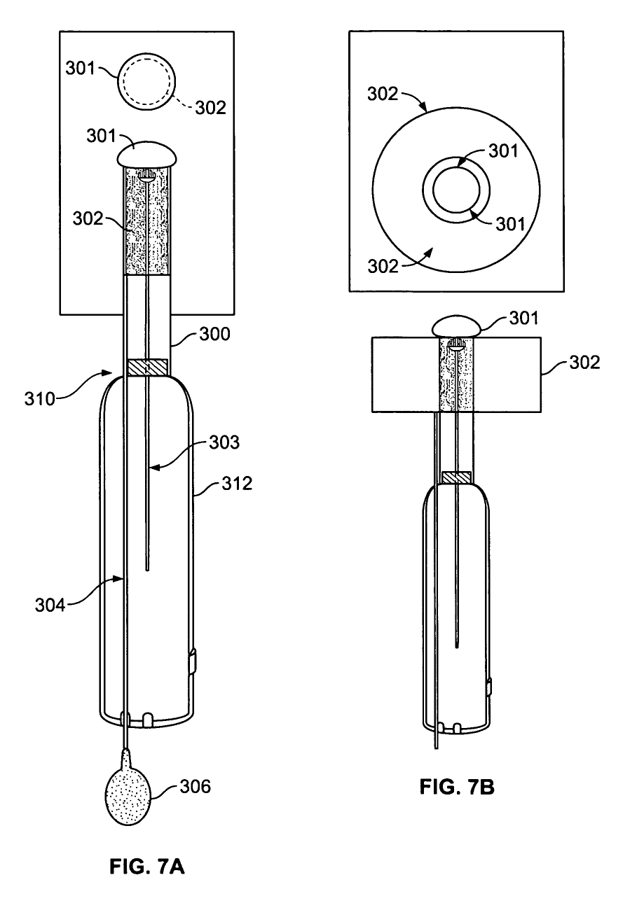

FIGS. 7A and B each illustrate side and top views of an operation of a probe 310 having a handle 312 and the tip 300 as described with reference to FIGS. 6A and B. In a closed position, the dilator 302 is deflated and fitted closely to the tip 300 to allow easy insertion and/or removal to and from the mammalian orifice or cavity. The conduit 304 is connected to a pressure source 306. Air, fluid or other pressure-providing fluid is injected into the dilator 301 from the pressure source 306 via the fluidic conduit 304. In an exemplary embodiment, the pressure source 306 is a manually-operated squeezable bulb connected to the handle 312 that stores a fluid, and when squeezed sends the fluid to the dilator 302 to inflate it.

As shown in FIG. 7B, when the dilator 302 is inflated to an open position, the dilator 302 expands the mammalian orifice or cavity to push back tissue therein, and create a larger opening for the probe 310. Accordingly, a larger surface area of the orifice or cavity is in view of the top 301 of the probe 310, and a camera provided to the top will have a wider angle of view.

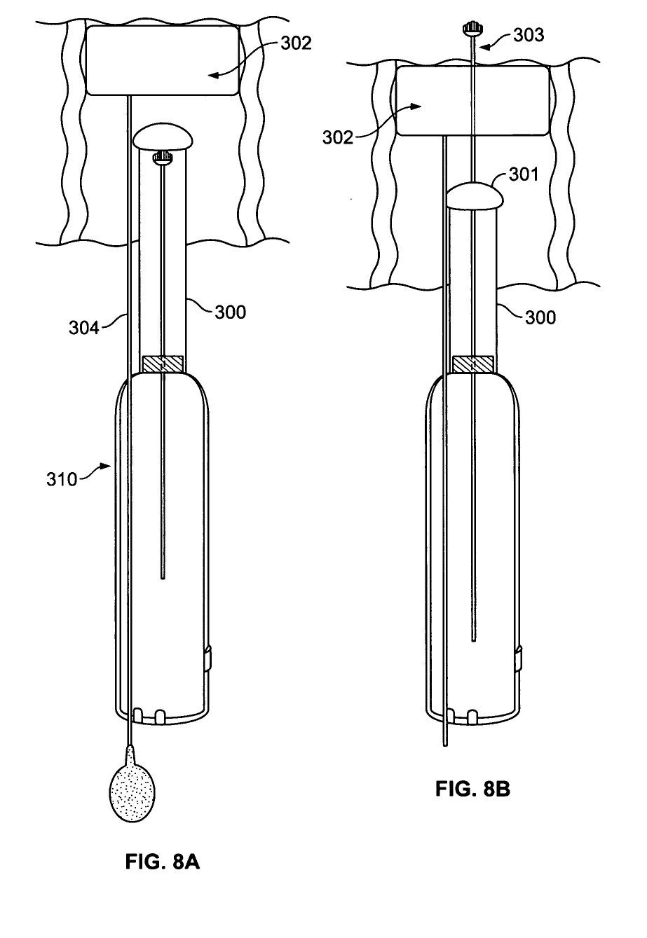

In one exemplary embodiment, a dilator in the open position can be slidably detached from the rest of the probe 310, as illustrated in FIGS. 8A and B. FIG. 8A illustrates the probe 310 being pulled back outwardly from the orifice or cavity, to increase the focal length of a field of view from a camera in the tip 300 or in general from the top of the tip 300, and the dilator 302 remains in place and stationary in the open position inside the orifice or cavity.

As shown in FIG. 8B, the probe 310 can be controlled and operated in various ways with the dilator 302 still connected to the conduit 304 and in the open position, but detached from tip 300 of the probe 310. In the configuration shown, the collection device 303 can be extended from the tip 300 to explore and gather tissue within the orifice or cavity. Once collection is done, the collection device 303 can be retracted back into the tip 300 and/or the handle 312 of the probe 310 to protect collected samples, shield the collection device 303 from damage, or the like.

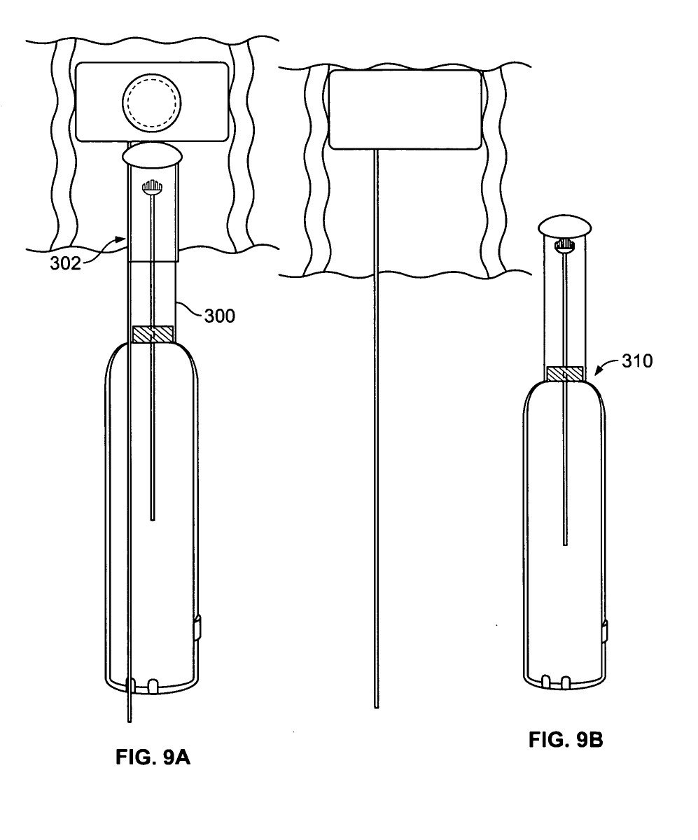

FIGS. 9A and B show further operation of the probe 310, illustrating several removal techniques of the probe 310 from the orifice or cavity. As shown in FIG. 9A, the dilator 302 is returned to the closed position, i.e. deflated and against the tip 300, such that the tip 200 of the probe 310 can be safely and comfortably removed from the orifice or cavity. Alternative, as shown in FIG. 9B, the dilator 302 can be kept in the open position, i.e. inflated, and then detached entirely or at least partially from the probe 310, so that the probe 310 can be removed from the expanded orifice or cavity.

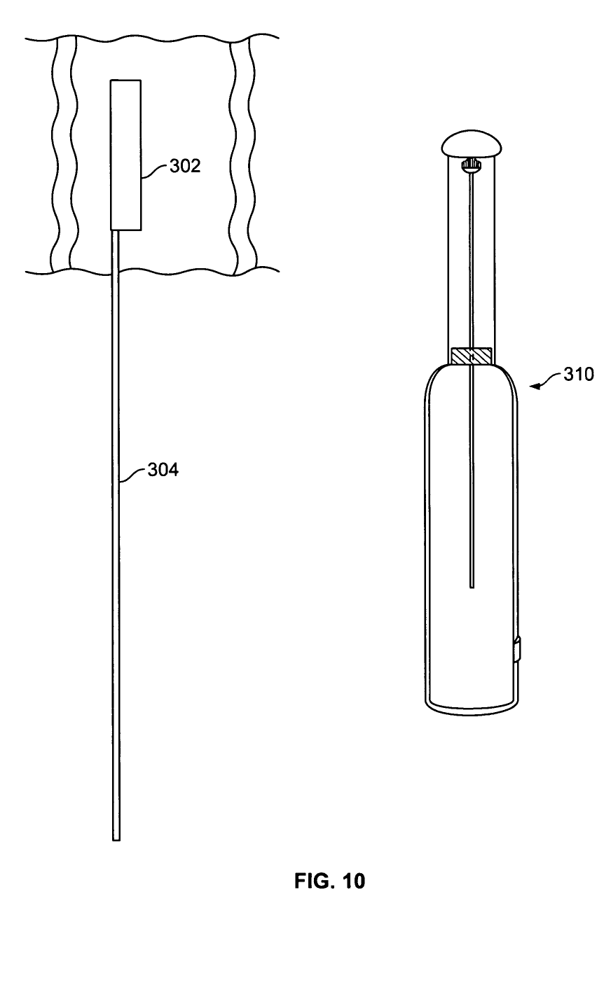

FIG. 10 illustrates a removal technique of the dilator 302 from the orifice or cavity apart from the probe 310. The dilator 302 is deflated by removing fluidic pressure from the dilator 302 back through the conduit 304. In one exemplary embodiment, the conduit 304 can include a clamp to close off the conduit and/or dilator 302 to maintain the dilator in a pressurized, inflated open position. The clamp can be opened to reopen the conduit 304 and/or dilator 302 to release the pressure in the dilator 302. Once the pressure is released and the dilator is substantially back to the closed, deflated position, the dilator 302 may be removed from the orifice or cavity. In an exemplary embodiment, the dilator 302 can be moved and controlled by a person pulling on the conduit 304 to move the dilator 302.

Although a few embodiments have been described in detail above, other modifications are possible. Other embodiments may be within the scope of the following claims.

1. A probe for an orifice or cavity of a mammal, the probe comprising:
a handle;
a tip coupled to the handle and including a video camera; and
an exciter coupled to the tip and configured to provide electric stimuli to tissue within the orifice or cavity.

2. A probe in accordance with claim 1, wherein the exciter includes a sheath around the tip.

3. A probe in accordance with claim 2, wherein the sheath includes one or more electrodes.

4. A probe in accordance with claim 1, further comprising circuitry to control the electric stimuli.

5. A probe in accordance with claim 1, wherein the exciter includes a vibrator.

6. A probe in accordance with claim 5, further comprising circuitry to control a vibration energy provided by the vibrator.

7. A probe in accordance with claim 6, wherein the circuitry is contained within the handle.

8. A probe for an orifice or cavity of a mammal, the probe comprising:
a handle;
a tip coupled to the handle and including a video camera; and
a dilator coupled to the tip and configured to open an interior wall of the orifice or cavity.

9. A probe in accordance with claim 8, wherein the dilator includes a plurality of slats configured to extend from a closed position against the tip to an open position extending from the tip.

10. A probe in accordance with claim 8, wherein the dilator includes a balloon configured to extend from a closed deflated position to an open inflated position.

11. A probe in accordance with claim 8, further comprising circuitry to control the dilator.

12. A probe in accordance with claim 11, wherein the circuitry is contained within the handle.

13. A probe for an orifice or cavity of a mammal, the probe comprising:
a handle;
a tip coupled to the handle and including a video camera;
an exciter coupled to the tip and configured to provide electric stimuli to tissue within the orifice or cavity; and
a dilator coupled to the tip and configured to open an interior wall of the orifice or cavity.

14. A probe in accordance with claim 13, wherein the exciter includes a sheath around the tip.

15. A probe in accordance with claim 14, wherein the sheath includes one or more electrodes.

16. A probe in accordance with claim 14, wherein the sheath includes a vibrator.

17. A probe in accordance with claim 13, further comprising circuitry contained within the handle for controlling the exciter.

18. A probe in accordance with claim 13, further comprising circuitry contained within the handle for controlling the dilator.

19. A probe in accordance with claim 18, wherein the dilator includes a balloon that is inflatable and deflatable under control of the circuitry.

20. A probe in accordance with claim 13, wherein the dilator includes one or more extendable arms.
